(19) United States
(12) Patent Application Publication
(10) Pub. No.: US 2025/0230408 A1
(43) Pub. Date: Jul. 17, 2025

(54) METHODS AND COMPOSITIONS FOR OPTOGENETICALLY ENGINEERED CELLS FOR NEURAL REPAIR

(71) Applicants: DREXEL UNIVERSITY, Philadelphia, PA (US); THE J. DAVID GLADSTONE INSTITUTES, San Francisco, CA (US)

(72) Inventors: Michael Aron Lane, Philadelphia, PA (US); Todd C. McDevitt, San Francisco, CA (US); Lyandysha Viktorovna Zholudeva, Concord, CA (US)

(21) Appl. No.: 18/708,200

(22) PCT Filed: Nov. 8, 2022
(86) PCT No.: PCT/US2022/079499

§ 371 Date: May 7, 2024

Related U.S. Application Data
(60) Provisional application No. 63/277,031, filed on Nov. 8, 2021.

(51) Int. Cl. C12N 5/0797; A61K 35/30

(52) U.S. Cl. CPC C12N 5/0623; A61K 35/30; C12N 2500/38; C12N 2501/13; C12N 2501/15; C12N 2501/41; C12N 2501/42; C12N 2501/727; C12N 2506/45; C12N 2510/00

(57) ABSTRACT

Compositions and methods for administering optogenetically-activatable cells for repair of the nervous system after injury or disease are contemplated herein.

### CROSS REFERENCE TO RELATED PATENT APPLICATIONS

This patent application claims the benefit of U.S. Provisional Patent Application No. 63/277,031, filed Nov. 8, 2021. The entire contents of the foregoing application are incorporated herein by reference, including all text, tables, drawings, and sequences.

### STATEMENT REGARDING GOVERNMENT FUNDING

This invention was made with government support under Grant numbers NS119348-01A1, NS081112, and NS116404 awarded by the National Institutes of Health. The government has certain rights in the invention.

### FIELD OF THE INVENTION

This invention relates to the fields of optogenetically-activatable cells for repair of the nervous system after injury or disease.

### BACKGROUND OF THE INVENTION

The nervous system is a complex highly specialized network that organizes explains, and direct interactions between the person and the world. The nervous system controls a variety of voluntary and involuntary functions. Accordingly, damage to the nervous system causes a variety of problems depending on the area that is damaged. Damage to the nervous system can occur slowly and cause a gradual loss of function or may be caused by a sudden and life-threatening problem. Symptoms are often severe; however, options for treating this damage are limited.

In decades of transplantation work, many different types of cells have been transplanted without having control over them after they are transplantation. These techniques rely on spontaneous mechanisms of action from the transplant to cause a therapeutic effect. No other invention has been able to demonstrate direct activation of cells that have been transplanted into a pathological environment. Thus, there is a need for safe and effective pharmacologic treatments capable of transplanting and activating cells for the repair of an injured or diseased nervous system.

### SUMMARY OF THE INVENTION

The present invention comprises engineered, neural progenitor cells having a response to an optogenetic stimulus that increases or decreases cell activity. In certain embodiments the stimulus is light on the visible light spectrum. In certain embodiments, the light is ultraviolet or infrared light. In another embodiment, the light is blue, red, or infrared. In certain embodiments, the response to light is reversable.

In another aspect, the present invention comprises methods for repairing a damaged nervous system in a patient in need thereof, the method comprising administration of the cells disclosed herein and activating the response of said cells. In certain embodiments, the damage to the nervous system is caused by injury or disease.

In still another aspect, the present invention comprises, methods for treating nervous system injury or disease in a patient in need thereof, the method comprising administering the cells disclosed herein and activating the response of said cells. In certain embodiments, the patient has damage to at least one of the brain or the spinal cord. In certain embodiments, the cells are obtained from at least one of the patient or a third-party donor and altered to have said response. In another aspect, the cells are obtained as optogenetic stem cells and differentiated to neural progenitor cells. In yet another aspect, administration of the cells improves motor recovery of the patient when compared to an untreated control. In certain embodiments, the cells display at least one of anatomical and functional evidence of connectivity.

In another aspect, administration of said cells occurs prior to surgical treatment. In yet another aspect, administration of said cells occurs after surgical treatment. In certain embodiments, the present invention further comprising said patient participating in rehabilitative physical therapy. In certain embodiments, the rehabilitative physical therapy maintains or strengthens muscle function or redevelops fine motor skills. In certain embodiments, the cells are activated weeks or months after administration.

In still another aspect, the present invention comprises in vitro methods of generating a neural progenitor cell comprising a) obtaining hPSCs from a donor; b) plating said hPSCs on mTeSR media and contacting said cells with CHIR and at least one Dual SMAD inhibitor (SMADi) and incubating said cells for about 5 days; c) replating the cells on Neural Induction Media and contacting said cells with CHIR, at least one Dual SMADi, and RA and incubating said cells for about 2 days; d) contacting the cells with RA, DAPT, and Pur(V2a) and incubating said cells for about 10 days; and e) replating the cells on BrainPhys Media and contacting said cells with G/BDNF, IGF, and CNTF to produce optogenetic neural progenitor cells. In certain embodiments, the neural progenitor cell is a V2a spinal interneuron. In certain embodiments, the cells of step b) and step c) are contacted with 0-6 μM, inclusive, of CHIR. In another aspect, the cells of step b) and step c) are contacted with 0, 2, 4, or 6 μM, of CHIR. In certain embodiments, the cells of step c) and step d) are contacted with 100 nM of RA. In another aspect, the cells of step d) are contacted with 1 μM of DAPT. In another aspect, the cells of step d) are contacted with 1 mM of Pur(V2a).

Still other aspects and advantages of these compositions and methods are described further in the following detailed description of the preferred embodiments thereof.

### BRIEF DESCRIPTION OF THE FIGURES

FIGS. 1A-1J. Optogenetic control of IN networks. Timeline for differentiating V2a spinal interneurons from hPSCs (FIG. 1A). Engineered human V2a interneurons positive for Chx10, (a transcriptional factor specific to V2a INs, FIG. 1B) and Tuj1 (a general marker for neurons, FIG. 1C) with DAPI (a stain used to label nuclei of all cells, FIG. 1D) with overlay depicted in (FIG. 1E). These cells can be differentiated from a channelrhodopsin hPSC line (FIG. 1F, FIG. 1G) and can be plated onto a microelectrode array (MEA, FIG. 1H) for electrophysiological characterization of neuronal activity (FIG. 1I) that is controllable with blue light stimulation (FIG. 1J, stimulation depicted with vertical lines, top). The MEA platform allows for both stimulation and recording of neuronal activity, allowing for optimization of stimulation protocol to elicit rhythmic patterns.

FIG. 2 Representative image of pretreated damaged spinal cord showing that the spinal cord does not regenerate.

FIG. 3. Engineering spinal interneurons from human induced pluripotent stem cells with representative images from Days 0, 1, 3, 5, 10, and 17.

FIG. 4 Phenotypic characterization of human spinal interneurons

FIG. 5 Representative images showing long distance growth of transplanted cells after 3 months.

FIG. 6 Representative images showing host to transplant innervation 3 months after transplant.

FIG. 7 Representative images showing donor to host connectivity 2 months after transplant.

FIG. 8A-8O Donor to host functional connectivity. Schematic of the experimental design (FIG. 8A) to demonstrate functional synaptic connectivity of transplanted human spinal interneurons (SpINs) to the phrenic motor circuit (spinal network that controls the diaphragm muscle). High magnification images at the transplant epicenter showing donor cells (ChR2-YFP, green, FIG. 8B), differentiate into neurons (NeuN, FIG. 8C), and some become synaptically integrated with phrenic circuit (indicated as PRV+, FIG. 8D), merge in (FIG. 8E). White arrows point to transplanted human SpINs that synaptically integrated with the phrenic circuit. Transplanted cells also extend their neurites directly to host phrenic motor neurons in the injured spinal cord (FIG. 8F-8H, merge in FIG. 8I). Examples of electrophysiological activity recorded from transplant (multi-unit recording), dorsal and ventral parts of the ipsilateral-to-injury hemidiaphragm at baseline (FIG. 8J), during blue light stimulation (FIG. 8K), and activity immediately post stimulation (FIG. 8L). Quantification of these electrophysiological data reveal that multi-unit activity from the transplant significantly increases during light-stimulation and remains elevated as tonic activity (FIG. 8M). Relative change of averaged diaphragm activity (40 second recordings, 3-5 minutes post blue light stimulation) from injured vehicle-controls and transplant recipients demonstrates elevated activity from transplant recipients both from the dorsal (FIG. 8N) and ventral (FIG. 8O) hemidiaphragms.

### DETAILED DESCRIPTION OF THE INVENTION

In certain embodiments of this invention, the use of optogenetically-activatable cells (e.g., neural cells) for repair of the nervous system (e.g., brain and spinal cord) after injury or disease are described. Optogenetically-activatable cells refers to cells that are responsive (e.g., increase or decrease their activity) to light (electromagnetic energy such as blue, red, infrared). A novel strategy for neural repair that involves transplanting donor neural progenitor cells engineered from optogenetic stem cells is disclosed herein. Doing so means that the cells can be transplanted as neural progenitor cells, survive the inhibitory environment known to comprise within the injured central nervous system, then weeks to months after transplantation, the cells can be controlled (activated or silenced) with light to elicit function from the transplant.

Emerging technologies, such as cell-based repair strategies, offer new promise for some of the most devastating medical conditions that currently lack treatments. However, to harness the full therapeutic potential of stem cells, it will be necessary to understand how to direct their differentiation to appropriate cell phenotypes and ensure that their phenotype and function persist after transplantation into a pathologic environment. Using pre-clinical spinal cord injury (SCI) as a testbed, we tested the therapeutic potential of transplanted human induced pluripotent stem cell (hiPSC)-derived spinal interneurons (SpINs). Neuroanatomical tracing and immunohistochemistry were used to assess transplant integration and connectivity with injured host tissue. Optogenetic activation of hiPSC-SpINs was used to assess development of synaptic connectivity to injured host circuits with time (1-, 2- and 3-months post-transplantation). Bilateral terminal diaphragm electromyography was used to assess functional contribution of transplanted human SpINs to the recovery of phrenic function with time (1-, 2- and 3-months post-transplantation). These studies demonstrated that transplanted human SpINs survived and integrated with injured cervical spinal cord circuits, displayed anatomical (e.g., pre- and post-synaptic proteins) and functional (intra-transplant and intra-spinal cord recordings) evidence of connectivity. Ongoing work is focused on silencing transplanted hiPSC-SpINs to further elucidate mechanisms by which transplanted cells may contribute to motor recovery post-SCI.

This invention and the use of it to repair the injured or diseased central nervous system is novel and gives precise control over when the transplanted cells are active and/or silenced. This invention gives us the control over the transplants themselves.

In certain embodiments, the invention is a biological product intended for transplantation into the injured or diseases nervous system that can be reversibly controlled with light. This ability enables the entrainment of transplanted cells to perform in a functionally and therapeutically relevant way. These cells are a specific type of neuron that are engineered to be relevant to neural repair.

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, where compositions, components, methods, or steps are described as required in one or more embodiments, additional embodiments are contemplated and are disclosed hereby for fewer compositions, components, methods, or steps, and for fewer compositions, components, methods, or steps in addition to other compositions, components, methods, or steps. All compositions, components, methods, or steps provided herein may be combined with one or more of any of the other compositions, components, methods, or steps provided herein unless otherwise indicated.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

A “sample” refers to a sample from a subject that may be tested. The sample may comprise cells, and it may comprise body fluids, such as blood, serum, plasma, cerebral spinal fluid, urine, saliva, tears, pleural fluid, and the like.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term does not denote a particular age or sex. Thus, adult and newborn subjects, as well as fetuses, whether male or female, are intended to be covered.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

The term “pharmaceutically acceptable carrier” or “diluent” is intended to include any and all solvents, dispersion media, coatings, antibacterial and antifungal agents, isotonic and absorption delaying agents, adjuvants and the like, compatible with administration to humans. In one embodiment, the diluent is saline or buffered saline.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

“Human pluripotent stem cells” or “hPSCs”, which include human embryonic stem cells (hESCs) and human induced pluripotent stem cells (hiPSCs), are cells that can self-renew indefinitely in culture while maintaining the ability to become almost any cell type in the human body. In certain embodiments, the hPSCs are genetically altered to respond an optogenetic stimulus. These optogenetic cells are then differentiated into optogenetic neural progenitor cells.

“Neural progenitor cells (NPCs)” are the progenitor cells of the CNS that give rise to many, if not all, of the glial and neuronal cell types that populate the CNS. NPCs do not generate the non-neural cells that are also present in the CNS, such as immune system cells. NPCs are present in the CNS of developing embryos but are also found in the neonatal and mature adult brain, and therefore are not strictly embryonic stem cells. NPCs are characterized based on their location in the brain, morphology, gene expression profile, temporal distribution and function. In general, embryonic NPCs have more potential than NPCs in the adult brain. In certain embodiments, the NPC is a V2a spinal interneuron.

V2a neurons are a genetically defined cell class that forms a major excitatory descending pathway from the brainstem reticular formation to the spinal cord. Their activation has been linked to the termination of locomotor activity based on broad optogenetic manipulations. V2a interneurons are essential to both forelimb and hindlimb movements, and two major types have been identified that emerge during development: type I neurons marked by high Chx10 form recurrent networks with neighboring spinal neurons and type II neurons that downregulate Chx10 and project to supraspinal structures. Types I and II V2a interneurons are arrayed in counter-gradients, and this network activates different patterns of motor output at cervical and lumbar levels.

V2a interneurons are present at all spinal levels, and anatomical and physiological studies have identified ascending connections to brainstem nuclei and local connections to motor neurons. These interneurons can be identified by their expression of the transcription factor Chx10/Vsx2.

In the ventral spinal cord, V2a interneurons represent locally projecting, candidate pre-motoneurons, involved in the control of left-right coordination of limb movements. In the brainstem, V2a neurons populate all anterio-posterior levels of the medullary and pontine reticular formations (RFs) and might have diverse functions and projection profiles. Initially linked to breathing control through local projections in the RF, they also represent a subset of excitatory reticulospinal (RS) neurons that collectively innervate multiple spinal segments. Broad optogenetic activation of V2a neurons in the gigantocellular reticular nucleus (Gi) causes an arrest of ongoing locomotion, while broad silencing favors mobility. These effects were attributed to V2a neurons with direct projections to the hindlimb controllers in the lumbar spinal cord. In certain embodiments, the engineered cells are V2a neurons, or V2a neuron-like cells.

“Optogenetics” refers to a biological technique that is used to control the activity of neurons and other cell types with electromagnetic energy or light. “Optogenetic cells” are cells that have a specific response to exposure to electromagnetic energy or light. In certain embodiments, the optogenetic cells only have a response to a specific wavelength of electromagnetic energy or a specific range of wavelengths of electromagnetic energy. In certain embodiments, hPSC are genetically engineered to respond to exposure to electromagnetic energy or light and differentiated into NPCs. In certain embodiments, the hPSCs are differentiated into NPCs which are genetically engineered to respond to exposure to electromagnetic energy or light.

“Electromagnetic energy” or “electromagnetic radiation” is a form of energy that is reflected or emitted from objects in the form of electrical and magnetic waves that can travel through space. This energy includes a broad spectrum from radio waves to gamma rays. This includes without limitation, infrared light, visible light, and ultraviolet light.

The nervous system is the major controlling, regulating and communication system in the body. It controls complicated processes like movement, though and memory and plays an essential role in natural bodily processes. The nervous system affects every aspect of health including, without limitation, thoughts, memory, learning, feelings, movements, balance, coordination, senses, including sight, hearing, taste, touch and feeling, sleep, healing, aging, heartbeat, breathing patterns, stress responses, digestion, and other body processes. The nervous system uses specialized cells called neurons to send signals throughout the body. These signals carry data to and from the brain.

A “damaged nervous system” revers to any disorder or condition that cases an injured nerve. “Injured nerves” refer to any nerve that has trouble sending or receiving a signal. In certain embodiments, the nerves are so damaged that they are completely unable to send or receive signals. Nerve injury can cause numbness, pain, inability to move, and/or death. Other symptoms include, vision problems, headaches, slurred speech, loss of sensation, tremors or tics (random muscle movements), changes in behavior or memory, problems with coordination or movement, and muscle weakness. The nervous system can be damaged by disease, such as cancers, autoimmune diseases like diabetes, lupus and rheumatoid arthritis, multiple sclerosis, stroke, accidental injury, pressure on the nerve, toxic chemicals including certain medicines, and advanced age. In certain circumstances, surgery is required to rectify the damage to the nervous system.

The phrase “TeSR media” refers to any medium that can be used to maintain hPSC cultures. Adjustments to a TeSR media will cause differentiation of the hPSC.

The phrase “CHIR” refers to the GSK3β inhibitor CHIR99021, 6-[[2-[[4-(2,4-Dichlorophenyl)-5-(5-methyl-1H-imidazol-2-yl)-2-pyrimidinyl]amino]ethyl]amino]-3-pyridinecarbonitrile. CHIR99021 is an aminopyrimidine derivative. CHIR99021 promotes self-renewal potential of embryonic stem cells (ESCs) of mice by inhibiting glycogen synthase kinase-3 (GSK-3) activity and potentiates the upregulation of β-catenin and c-Myc functions. CHIR99021 promotes self-renewal of ESCs by modulating transforming growth factor β (TGF-β), Notch and mitogen-activated protein kinases (MAPK) signaling pathways. CHIR99021 is an agonist of wingless/integrated (wnt) signalling, upregulates cyclinA expression and promotes cell proliferation in non-small-cell lung cancer (NSCLC) cell lines.

SMADs comprise a family of structurally similar proteins that are the main single transducers for receptors of the TFG-B superfamily, which are critically important for regulating cell development and growth. An “SMAD inhibitor” reduces or decreases the activity or expression of SMAD. Dual SMAD inhibition refers to a process using SMAD inhibitors to rapidly differentiate hPSCs into early neurectoderm.

“Neural Induction Media” refers to Gibco® PSC Neural Induction Medium or other similar commercially available serum-free medium that provides high efficiency neural induction of human pluripotent stems cells (PSCs).

A Notch Pathway is a highly conserved signaling pathway that is crucial in development and is implicated in malignant transformation. A Notch Pathway inhibitor reduces or decreases the activity of the Notch Pathway. In certain embodiments, the Notch Pathway inhibitor is DAPT.

“BrainPhys Media” refers to the neuronal medium developed by Bardy et al (BrainPhys supports neurophysiological activity, Bardy et al, Proceedings of the National Academy of Sciences May 2015, 112 (20) E2725-E2734; DOI: 10.1073/pnas. 1504393112) and available from Stemcell Technologies (cat #ST-05790). BrainPhys Media simulates various in vivo conditions including: inorganic salt concentration, glucose levels, and osmolarity. Additionally, BrainPhys Media allows for several Neuronal functions including spontaneous and evoked action potentials, spontaneous network calcium dynamics, excitatory synaptic activity, and inhibitory synaptic activity. To sustain cell survival and/or neural differentiation in vitro, supplements such as antioxidants, growth factors, hormones, and proteins can be added to basal media.
“G/BDNF” refers to brain derived neurotropic factor.
“IGF” refers to interferon gamma.
“CNTF” refers to Ciliary Neurotrophic Factor.

### Methods of Generating Neural Progenitor Cells

Provided herein are methods for generating optogenetically-activatable neural progenitor cells for use in the treatment of the nervous system in a subject in need thereof. By optogenetically-activatable it is meant that the cells engineered by this method respond to a stimulus that either increases or decreases their activity. This stimulus is electromagnetic energy or light. In certain embodiments this light is selected from a wavelength on the visible light spectrum, ultraviolet, or infrared light. In other embodiments, the light is blue, red, or infrared. In certain embodiments, the response to this stimulus is reversable and the cells can be activated or silenced at will. In certain embodiments, the cells are stimulated after several weeks or months.

The stimulus may be electromagnetic radiation, which spans an enormous range of wavelengths and frequencies. This range is known as the electromagnetic spectrum. The electromagnetic spectrum is generally divided into seven regions, in order of decreasing wavelength and increasing energy and frequency. The common designations are radio waves, microwaves, infrared (IR), visible light, ultraviolet (UV) light, X-rays and gamma-rays. Radio waves are at the lowest range of the electromagnetic spectrum, with frequencies of up to about 30 billion hertz, or 30 gigahertz (GHz), and wavelengths greater than about 0.4 inch (10 millimeters). Infrared is in the range of the electromagnetic spectrum between microwaves and visible light. IR has frequencies from about 30 to 400 THz and wavelengths of about 0.00003 to 0.004 inch (740 nanometers to 100 micrometers). Visible light is found in the middle of the electromagnetic spectrum, between IR and UV. It has frequencies of about 400 to 800 THz and wavelengths of about 0.000015 to 0.00003 inch (380 to 740 nanometers). Ultraviolet light is the range of the electromagnetic spectrum between visible light and X-rays. It has frequencies of about 8×10¹⁴ to 3×10¹⁶ Hz and wavelengths of about 0.0000004 to 0.000015 inch (10 to 380 nanometers).

When used herein as a stimulus, the electromagnetic radiation may be any wavelength on the electromagnetic spectrum. In certain embodiments, the wavelength is between 10 nanometers and 10 millimeters. Further ranges/subranges, and integers within those ranges/subranges, are also contemplated, including but not limited to wavelengths greater than 10 millimeters, wavelengths from 100 micrometers to 10 nanometers, wavelengths from 100 micrometers to 740 nanometers, wavelengths from 100 micrometers to 10 micrometers, wavelengths from 10 micrometers to 740 nanometers, wavelengths from 740 nanometers to 380 nanometers, wavelengths from 740 nanometers to 625 nanometers, wavelengths from 625 nanometers to 580 nanometers, wavelengths from 580 nanometers to 565 nanometers, wavelengths from 565 nanometers to 520 nanometers, wavelengths from 520 nanometers to 500 nanometers, wavelengths from 500 nanometers to 430 nanometers, wavelengths from 430 nanometers to 380 nanometers, wavelengths from 740 nanometers to 430 nanometers, wavelengths from 740 nanometers to 500 nanometers, wavelengths from 740 nanometers to 520 nanometers, wavelengths from 740 nanometers to 565 nanometers, wavelengths from 740 nanometers to 580 nanometers, wavelengths from 625 nanometers to 380 nanometers, wavelengths from 580 nanometers to 380 nanometers, wavelengths from 565 nanometers to 380 nanometers, wavelengths from 520 nanometers to 380 nanometers, wavelengths from 500 nanometers to 380 nanometers, wavelengths from 380 nanometers to 100 nanometers, wavelengths from 380 nanometers to 315 nanometers, wavelengths from 315 nanometers to 380 nanometers, wavelengths from 280 nanometers to 100 nanometers.

Provided herein, in one aspect, is a method of generating optogenetically-activatable neural progenitor cells. In certain embodiments, the methods disclosed herein comprise generating neural progenitor cells from human pluripotent stem cells (hPSCs) obtained from a donor. In certain embodiments the donor is the same person that is treated using the method disclosed herein. In other embodiments, the donor is a healthy subject.

In some embodiments, the cells obtained from the donor are plated on mTeSR media and contacted with (CHIR) and at least one Dual SMAD inhibitor (SMADi). In certain embodiments, the SMADi is selected from LEFTY1, Noggin and SB431542. In other embodiments, the SMAD pathway is inhibited using more than one agent, such as SB431542 and dorsomorphin (DM). The cells are then incubated for about 5 days. In certain embodiments, the cells are incubated for about 1-10 days. Further ranges/subranges, and integers within those ranges/subranges, are also contemplated, including but not limited to about 1-5 days, about 1-6 days, about 1-7 days, about 1-8 days, about 1-9 days, about 5-10 days, about 5-9 days, about 5-8 days, about 5-7 days, about 5-6 days, about 3-7 days, about 4-6 days, at least 2 days, at least 3 days, at least 4 days, at least 5 days, at least 6 days, at least 7 days, at least 8 days, at least 9 days, at least 10 days.

The cells are then replated in Neural Induction Medium. Such neural induction media are known in the art, e.g., DMEM/F12, 1% N2 supplement (Thermo Fisher Scientific), 2 μg ml-1 heparin (Sigma-Aldrich) and 1% penicillin/streptomycin. The CHIR and SMADi remain present in the Neural Induction Media and the cells are further contacted with Retinoic Acid (RA). The cells are incubated on this media for about 2 days, then the CHIR and SMADi are removed and the cells are contacted with RA, a Notch Pathway Inhibitor, and purmorphamine (Pur). In certain embodiments the Notch Pathway inhibitor is DAPT. The cells are then allowed to incubate for about 10 days before being re-plated on BrainPhys Media (available from Stem Cell Technologies (cat #ST-05790)) before being administered to the patient. In certain embodiments, while on the BrainPhysMedia, the cells are contacted with G/BDNF, IGF, and CNTF.

In certain embodiments, the cells are replated on BrainPhys media after about 5-15 days. Further ranges/subranges, and integers within those ranges/subranges, are also contemplated, including but not limited to about 5-10 days, about 6-10 days, about 7-10 days, about 8-10 days, about 9-10 days, about 10-15 days, about 11-15 days, about 12-15 days, about 13-15 days, about 14-15 days, about 8-12 days, about 9-11 days, at least 2 days, at least 3 days, at least 4 days, at least 5 days, at least 6 days, at least 7 days, at least 8 days, at least 9 days, at least 10 days.

The person of skill in the art can produce cells according to the methods taught herein, and techniques known in the art, e.g., Brown, Chelsea R et al. “Generation of v2a interneurons from mouse embryonic stem cells.” Stem cells and development vol. 23,15 (2014): 1765-76. doi: 10.1089/scd.2013.0628, which is incorporated herein by reference.

### Methods of Treatment

Provided herein are methods of treatment of damage to the nervous system after injury or disease. The methods include administration of an effective amount of optogenetically-activatable neural progenitor cells to a subject in need thereof and activating the response of the cells. In certain embodiments, the patient has damage to the brain and/or the spinal cord. In some embodiments, treatment using the method provided herein improves motor recovery of the patient when compared to an untreated control. In certain embodiments, the cells display anatomical and/or functional evidence of connectivity. In certain embodiments, treatment further comprises at least one additional activation of the cell response.

In certain embodiments, the cells are generated by the method disclosed herein. In certain embodiments, the cells are obtained from a donor. In certain embodiments, the patient is the donor. In other embodiments, the donor is a healthy subject.

In certain embodiments, the method provided herein further comprises patient participation in rehabilitative physical therapy. In certain embodiments, the patient is treated after surgical treatment of the nervous system.

### Administration

The cells described herein may be delivered to a subject by any means. These cells may be administered alone, as pharmaceutical composition in combination with diluents and/or carriers and/or buffers, and other components. In one embodiment, they may be administered in saline. In another embodiment, in a hydrogel. Among other formulations, both saline and hydrogel formulations are contemplated for delivery by injection. Other components may include cytokines, cells, or other agents conventionally used to repair a damaged nervous system. Compositions may include stabilizers, antioxidants, and/or preservatives. Compositions may include, e.g., neutral buffered saline or phosphate buffered saline.

The compositions described herein can be formulated for intravenous (IV), intramuscular (IM), intra-articular (IA) and intrathecal (lumbar puncture) administration. The compositions can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

Carriers may include pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. Carriers also includes any and all coatings, antibacterial and antifungal agents, and absorption delaying agents, and the like that are compatible with the activity of the components, e.g., cells, to be delivered, and are physiologically acceptable to the patient. Supplementary active compounds may also be incorporated into the compositions. Pharmaceutically acceptable salt of the compound or molecule useful within the invention.

Other ingredients that may be included are excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Still other additional ingredients that may be included in the pharmaceutical compositions used in the practice of the invention and are known in the art and described elsewhere.

In certain embodiments, the cells described herein are delivered directly to the site of injury. In other embodiments, the cells are delivered via intrathecal administration. In other embodiments, the cells are delivered IV. In other embodiments, the cells are delivered intramuscularly or intra-arterially. The cells may be delivered in one administration, or in several administrations over a course of time.

Effective amounts of the neural progenitor cells and other aspects of a pharmaceutical composition may be determined by one of skill in the art, including by a physician. Such amounts may be determined by with consideration of the age and/or weight of a patient, and further by the size, condition, location, and/or severity of the wound or wounds to be treated. In one embodiment, administration of from 1 to 100 million cells is appropriate. Further ranges/subranges, and integers within those ranges/subranges, are also contemplated, including but not limited to from 1 to 10 million, 1 to 5 million, 1 to 1 million, 1 to 500,000, 1 to 400,000, 1 to 300,000, 1 to 250,000, 1 to 200,000, 1 to 150,000, 1 to 100,000, 1 to 50,000, 1 to 40,000, 1 to 30,000, 1 to 20,000, 1 to 10,000, 1 to 5,000, 1 to 2,500, 1 to 1,000, 1 to 100, 10 million to 100 million, 50 million to 100 million, 75 million to 100 million.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with a damaged nervous system.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

In certain embodiments, the cells described herein are provided with one or more additional therapies for the damaged nervous system. Treatment for includes medications such as those used to promote nerve cell regeneration or to improve the function of existing nerves.

In certain embodiments, the cells described herein are provided with one or more additional therapies for treating a damaged nervous system, including without limitation rehabilitative physical therapy.

Still further provided are kits comprising one of more of the cells described herein in one or more vials, tubes, or other suitable vessels. The kit may further comprise a syringe or other medical instrument suitable to deliver a composition to a subject.

### Optogenetic Activation of Cells

Provided herein are cells that are responsive to a stimulus. In certain embodiments the stimulus is electromagnetic energy such as visible light, ultraviolet light, infrared, blue or red. In certain embodiments, this light is delivered in vivo. In certain embodiments, the light is delivered using laser lights, LED lights, broadband light sources, or implantable microscopic inorganic LEDs (micro-ILEDs). In one embodiment, the light is an implantable optic fiber. See, e.g., Usseglip et al, Control of Orienting Movements and Locomotion by Projection-Defined Subsets of Brainstem V2a Neurons, Current Biology 30, 4665-4681 Dec. 7, 2020 which is incorporated herein by reference.

The duration for delivery of the light or electromagnetic radiation is long enough to activate the cells without damaging the surrounding tissues. In certain embodiments, the light or electromagnetic radiation is delivered for 1 millisecond-10 second durations. In certain embodiments, the stimulus is provided at the same time that the cells are administered. Further ranges/subranges, and integers within those ranges/subranges, are also contemplated, including but not limited to at least about 1-100 milliseconds, 100 milliseconds-1 second, 1-10 seconds, 2-8 seconds, 3-7 seconds, 4-6 seconds, and 5 seconds.

In certain embodiments, the stimulus is delivered multiple times after administration of the cells. In certain embodiments, the stimulus is administered at least 1, 2, 3, 4, 5, 6, 7, 8, 9, 10, or more times after the initial stimulus. In certain embodiments, the stimulus is administered one or more times per hour for several hours, days, weeks, or months. In certain embodiments, the stimulus is administered one or more times per day for several days, weeks or months.

The cells will be stimulated after the cells are administered to a patient. In certain embodiments, the stimulus is delivered from 15-21 days after the cells are administered. Further ranges/subranges, and integers within those ranges/subranges, are also contemplated, including but not limited to at least about 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, or 12 months after administration of the cells.

Other examples of light delivery systems are known by the skilled artisan. (See, e.g. Boyle, P. M., Karathanos, T. V., and Trayanova, N. A. (2018a). Cardiac optogenetics: 2018. JACC Clin. Electrophysiol. 4, 155-167. doi: 10.1016/j.jacep.2017.12.006)

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Differentiating V2A Spinal Interneurons from HPSCS

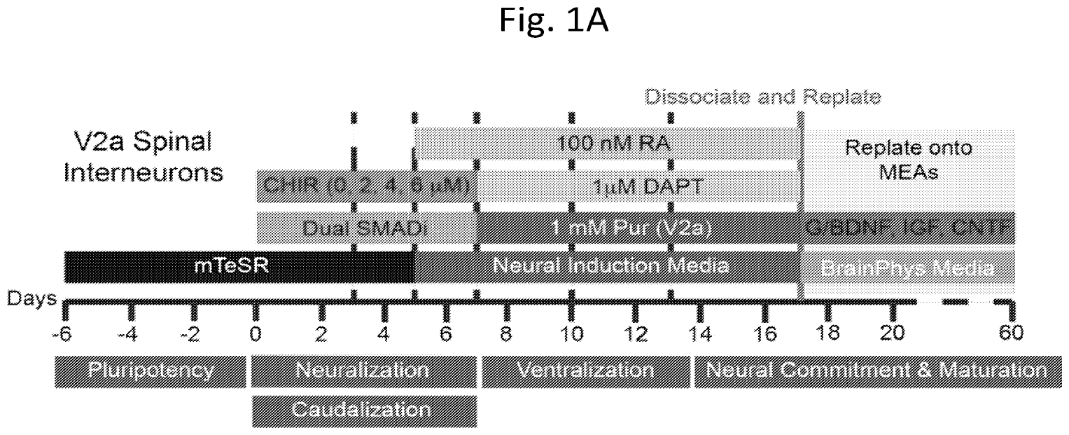

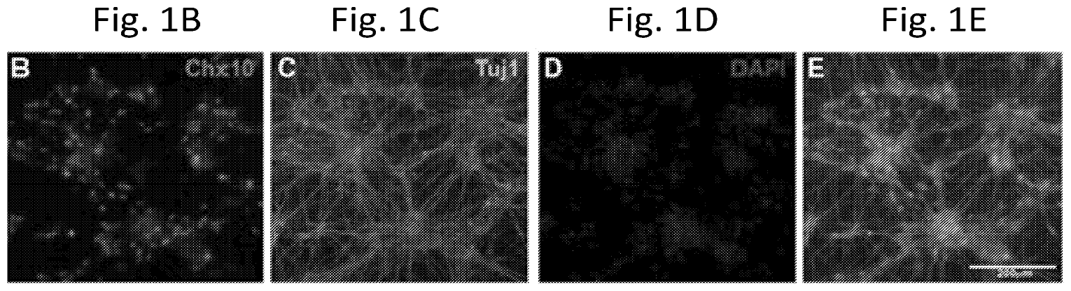

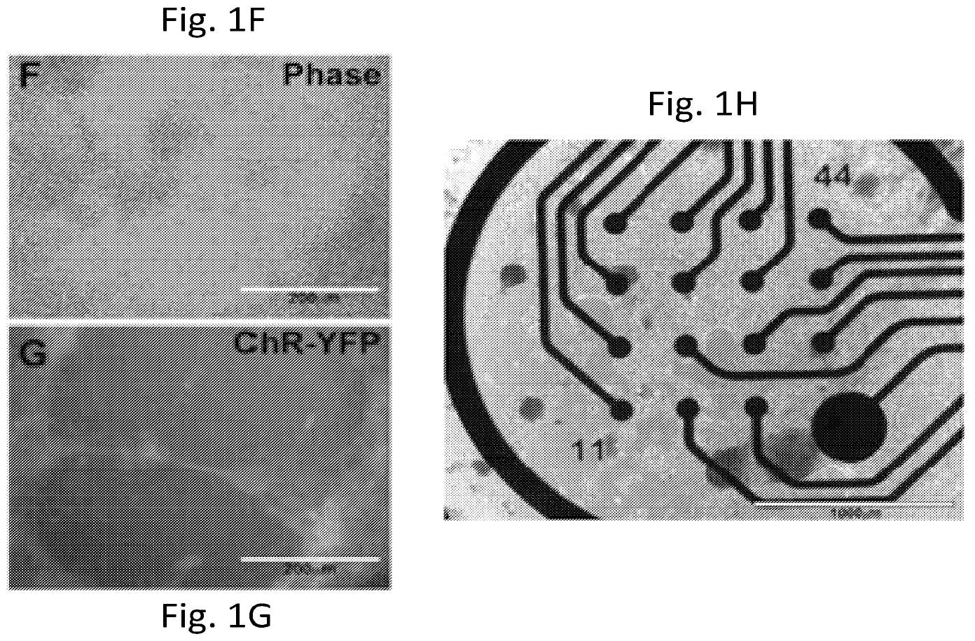

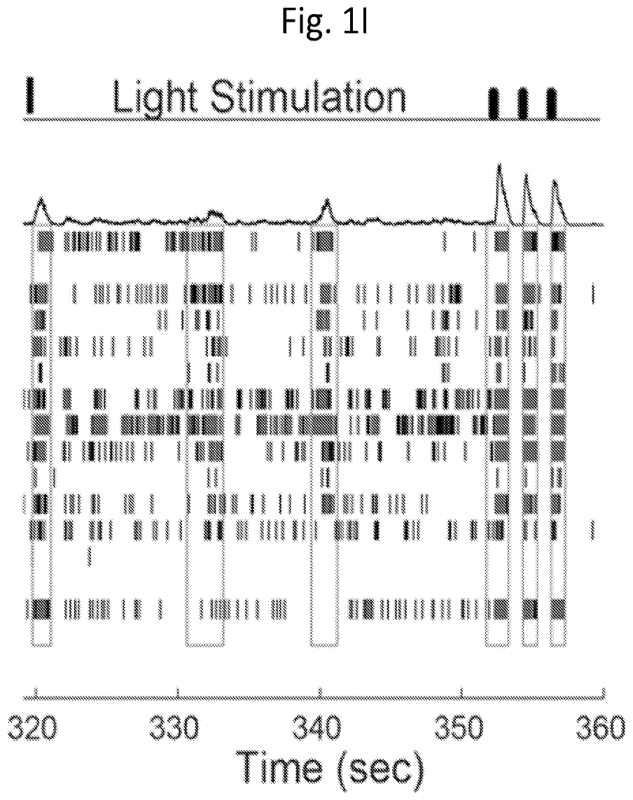

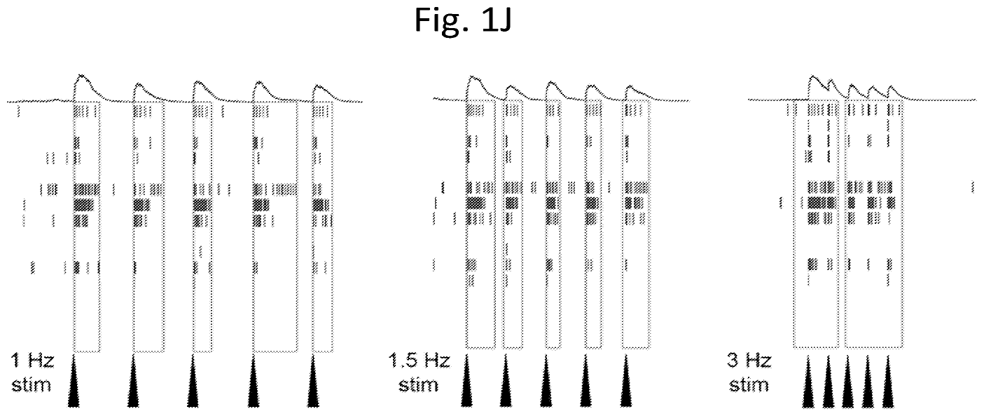

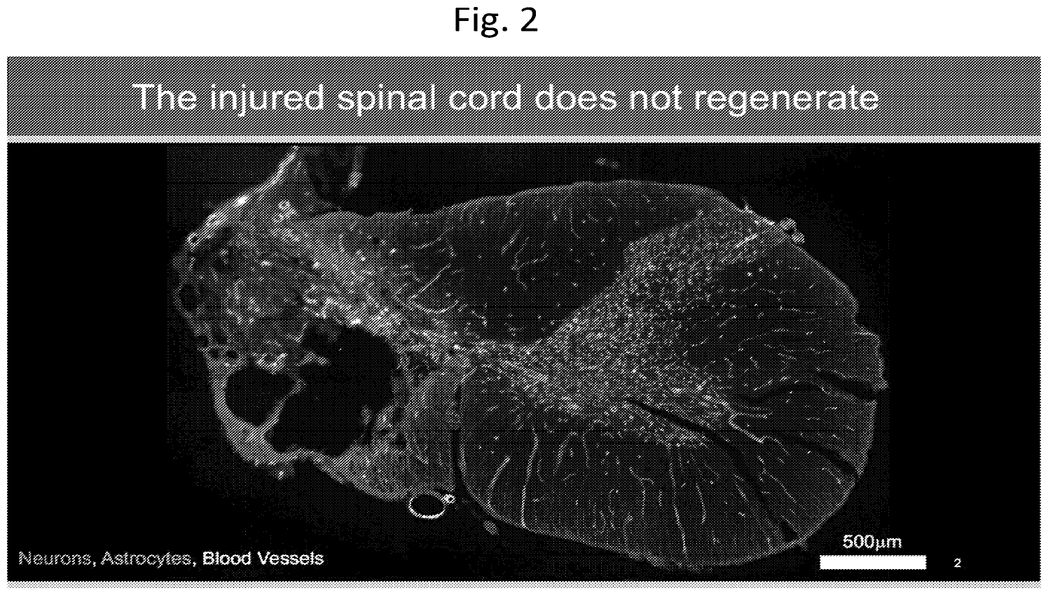

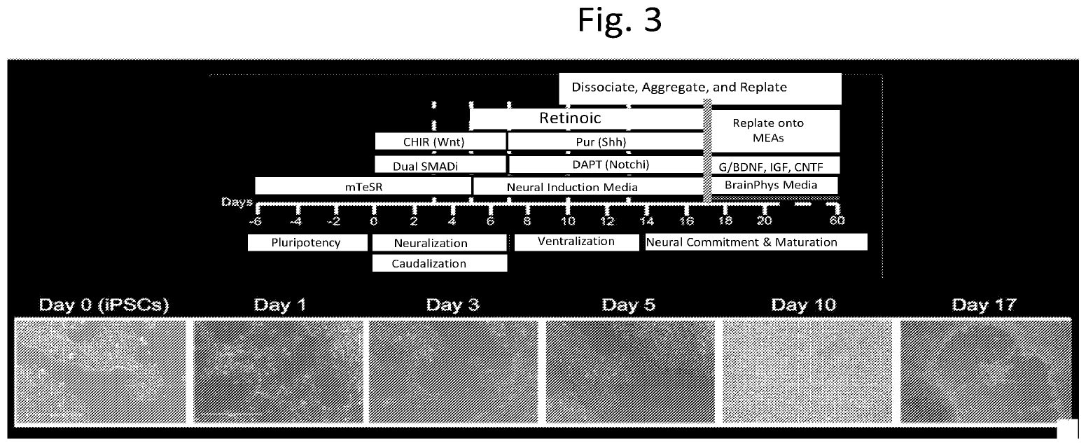

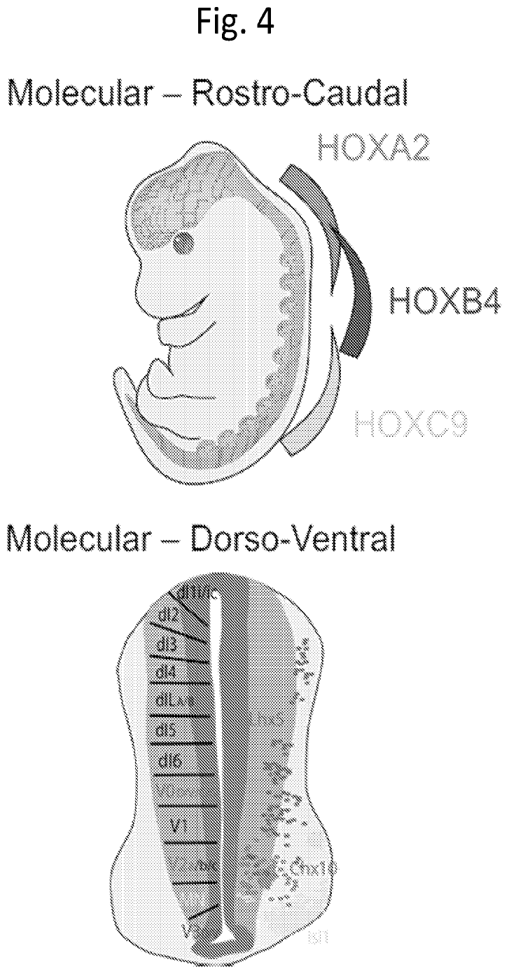

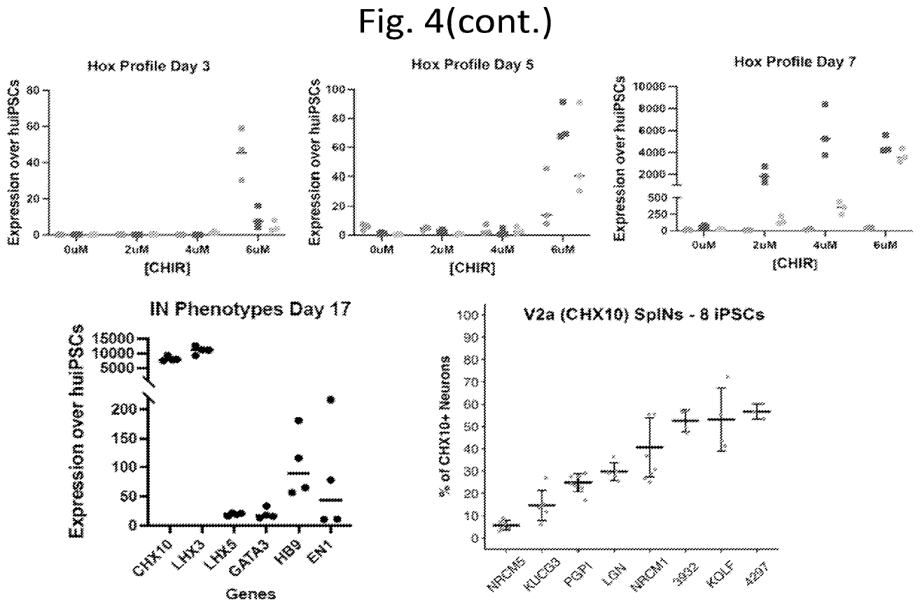

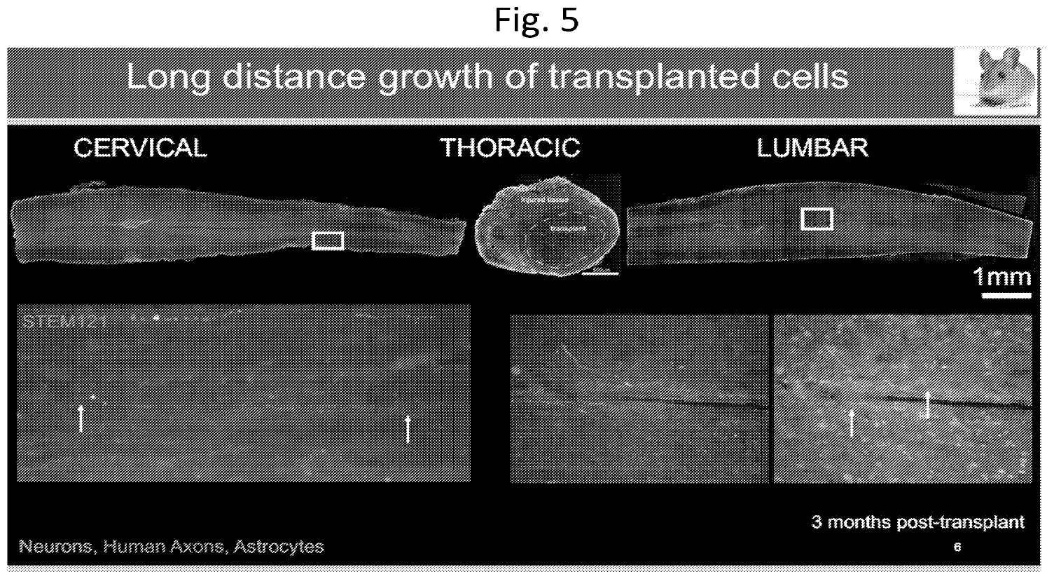

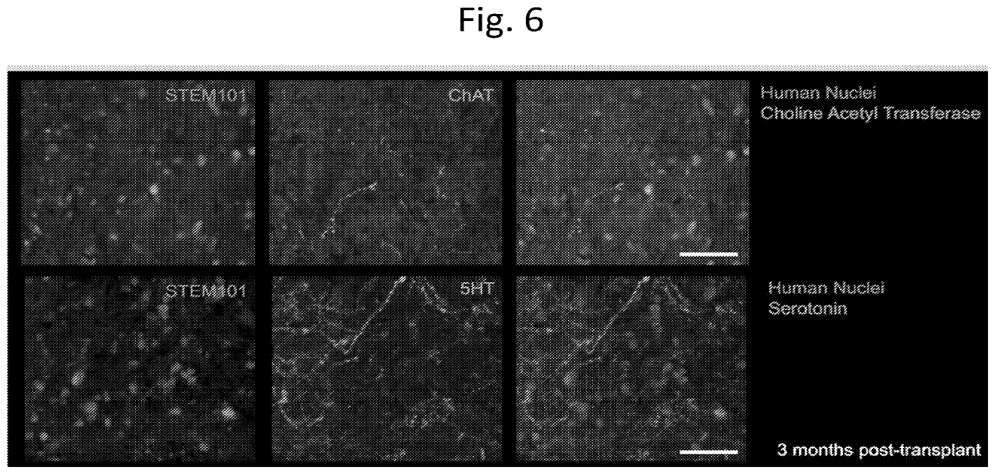

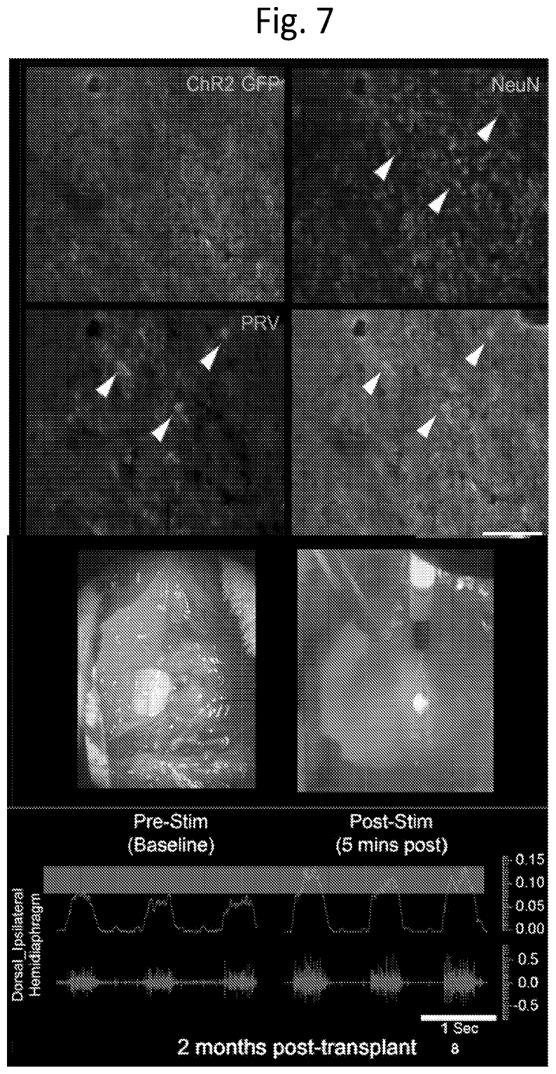

The timeline for differentiating V2a spinal interneurons from hPSCs is seen in FIG. 1A. Donor hPSCs are obtained from a third party and plated on mTeSR media. Cells are then contacted with at least one Dual Smad inhibitor (SMADi) and CHIR for 5 days. In certain embodiments, 0-6 μM, inclusive, of CHIR are added to the media. In certain embodiments, 0, 2, 4, or 6 μM of CHIR are added to the media.

At day 5, the cells are plated on Neural Induction Media and contacted with approximately 100 nM of Retinoic Acid (RA). In certain embodiments, the Neural Induction Media comprises Gibco® PSC Neural Induction Medium or other similar commercially available serum-free medium that provides high efficiency neural induction of human pluripotent stems cells (PSCs). On Day 7, the CHIR and Dual SMADi are removed and replaced with 1 μM of a Notch pathway inhibitor (DAPT) and 1 mM purmorphamine (Pur(V2a)). The cells remain on this media until day 17 where they are replated onto BrainPhys Media and contacted with G/BDNF, IFG, and CNTF. These cells can also be plated onto a microelectrode array (MEA) for further testing. In certain embodiments, the BrainPhys Media is the neuronal medium developed by Bardy et al (BrainPhys supports neurophysiological activity, Bardy et al, Proceedings of the National Academy of Sciences May 2015, 112 (20) E2725-E2734; DOI: 10.1073/pnas. 1504393112)

The cells were then tested for Chx10, a transcriptional factor specific to V2a Ins, and Tuj1, a general marker for neurons, and stained with DAPI. See FIGS. 1B-1D. These tests indicate successful engineering of optogenetic V2a spinal interneurons.

### Example 2: Optogenetic Analysis of V2A Cells

The cells obtained from Example 1 were plated onto a microelectrode array (MEA) and analyzed for electrophysiological characterization of their neuronal activity. See FIGS. 1H-1I. The MEA platform allows for both stimulation and recording of neuronal activity. This allows for optimization of stimulation protocol to elicit rhythmic patterns. FIG. 1J shows that neuronal activity of the engineered human V2a interneurons is controllable with blue light stimulation.

### Example 3: Administration and Activation of V2A Cells

Subjects are anesthetized, and the skull or spinal column is exposed. Engineered cells as generated in Example 1 are delivered using a pulled glass pipette connected to a syringe pump. The infusion flow is set to 100 nL/min. Sample coordinates (in mm) used to target V2a Gi neurons are: 6.0 from bregma, 0.8 lateral, and 4.5 from the dorsal brain surface (for a mouse). After the injection, the pipette is held in place for 5 min before being slowly retracted. For optogenetic activations, a 200 mm core 0.39 NA optic fiber (Thorlabs) connected to a 1.25 mm diameter ferrule (Thorlabs) is implanted ˜500 mm above the injection site. This operation is performed during the same surgery as the viral injection when both are targeted to the brainstem. For activating spinally-projecting V2a neurons, the spinal injection is performed first (see below) and the optic fiber may be implanted 5 to 7 days later. Dental cement (Tetric Evoflow) is used to secure the implanted ferrules. Animals are followed daily after the surgery.

Behavioral experiments start 15 to 21 days after the viral injection. Implanted animals are connected to a laser source (473 nm DPSS system, LaserGlow Technologies, Toronto, Canada) through a mating sleeve (Thorlabs). In all conditions except EMG recordings, light is delivered in trains of pulses of 15 ms at 40 Hz frequency for a duration of 500 ms. For EMG recordings we also use single pulses of 5 ms duration. We use the minimal laser power sufficient to evoke a response, which is measured to be between 5-12 mW at the fiber tip using a power meter (PM100USB with S120C silicon power head, Thorlabs) to restrict photo-activations unilaterally [64], prevent heat, and exclude an unintentional silencing by over-activation.

### Example 4: Analysis of Cell Functional Connectivity to Phrenic Circuit

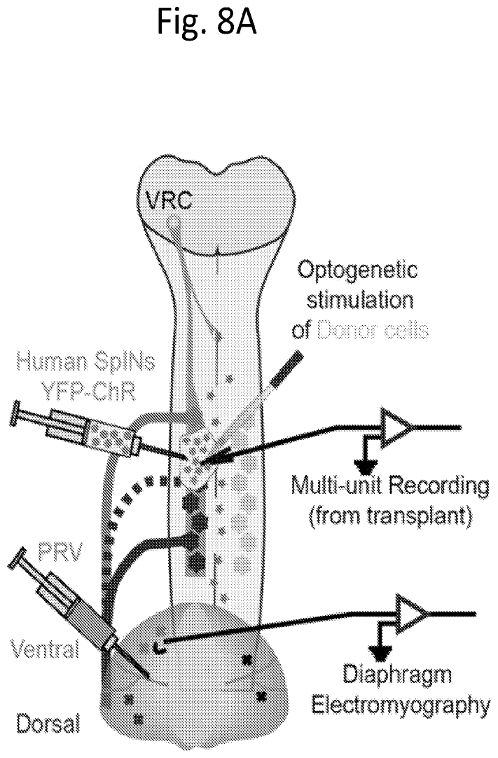

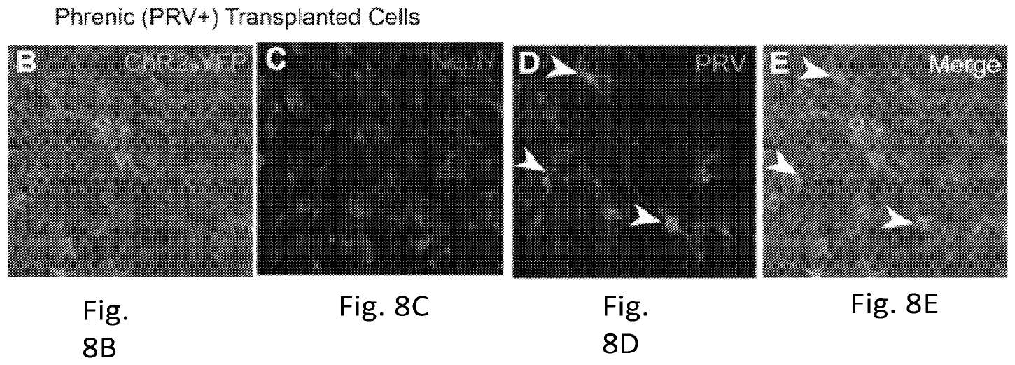

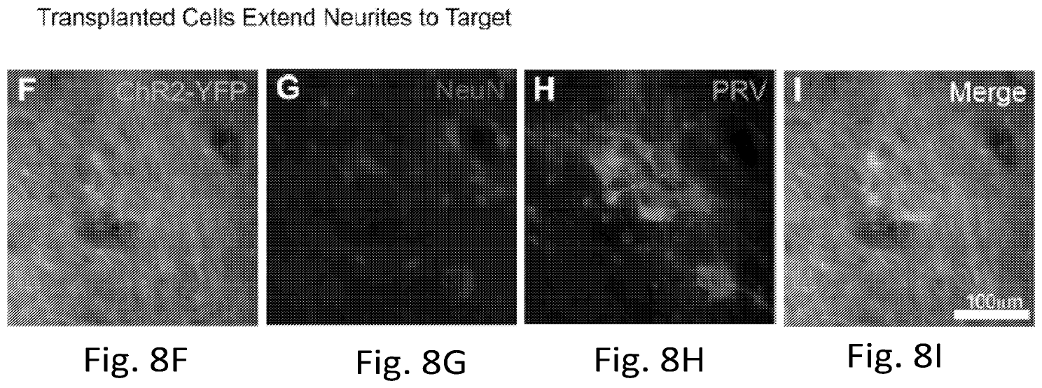

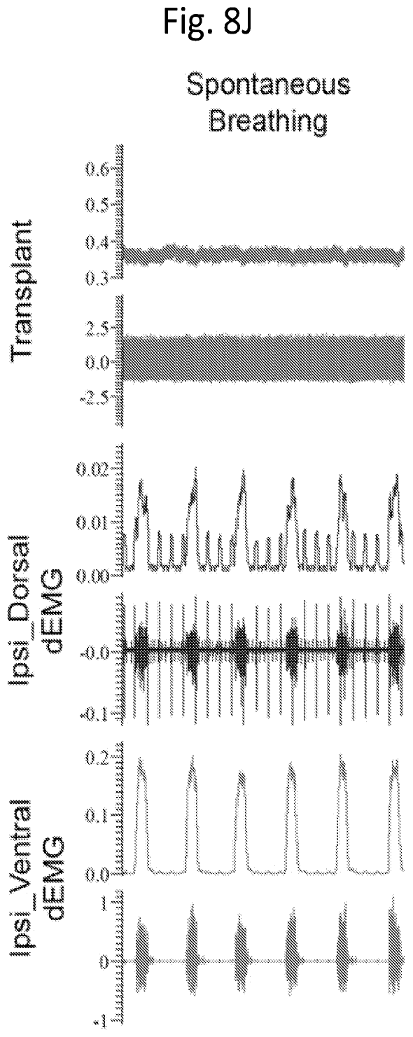

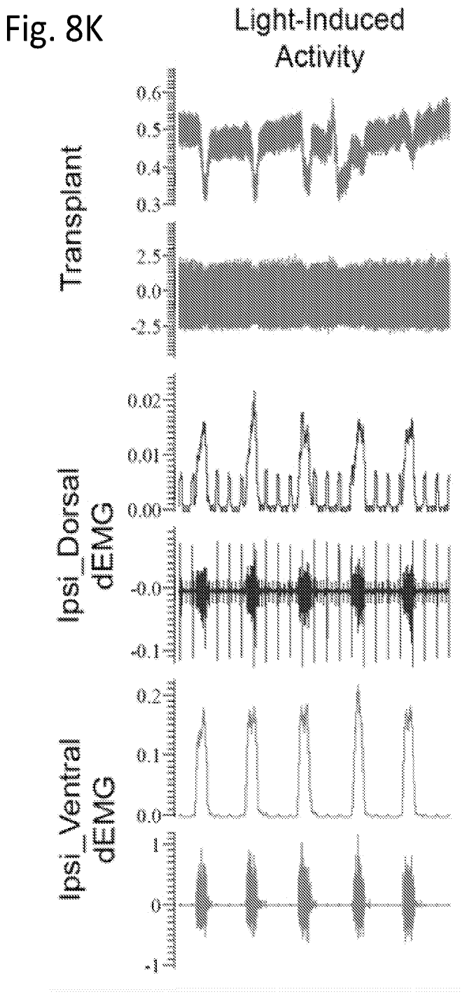

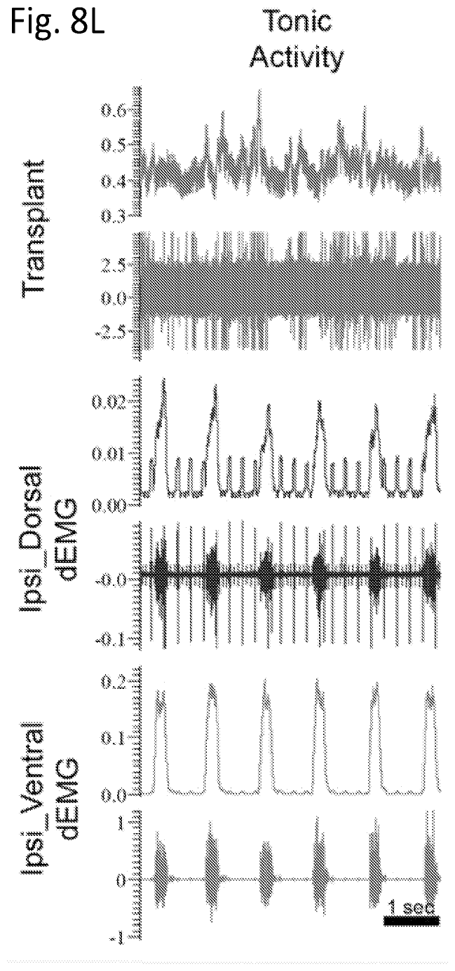

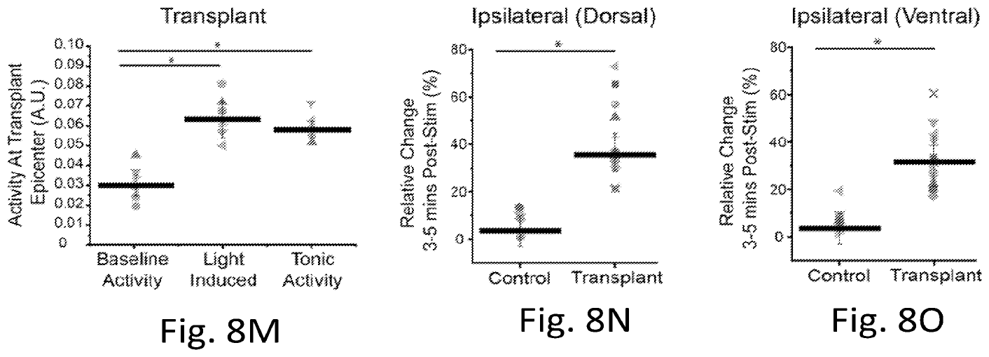

A graphical representation of this Example is shown in FIG. 8A. Human SpIN cells (huSpINs) were administered to the injured cervical spinal cord, at the location of the phrenic motor network (controlling the diaphragm muscle), and activated with optogenetic stimulation by blue light.

Electrophysiological activity of the transplanted cells was recorded at the transplant site, and the dorsal and ventral parts of the ipsilateral-to-injury hemidiaphragm during stimulation (FIG. 8K) and immediately after stimulation (FIG. 8L) and compared to a baseline reading (FIG. 8J). A forty second recording of these readings were quantified in FIGS. 8M-8O. Quantification of these electrophysiological data reveal that multi-unit activity from the transplant significantly increases during light-stimulation and remains elevated as tonic activity. This increase in activity is demonstrated at the transplant site, dorsal and ventral hemidiaphragms.

To determine functional synaptic connectivity of the transplanted huSpINs to the phrenic circuit, a sample was taken from the transplant epicenter and the phrenic circuit. High magnification (100 μm) images of the transplanted cells at the phrenic circuit show differentiation of the huSpINs into neurons. (FIG. 8C) These differentiated cells were further showed successfully synaptic integration into the phrenic circuit. (FIG. 8D) Additionally, images of the target site show that the successfully integrated cells extend their neurites to the target, phrenic motor neurons. (FIGS. 8F-8H) These images of the phrenic motor neurons provide further evidence of successful synaptic integration of the transplanted cells.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

1. An engineered, neural progenitor cell having a response to an optogenetic stimulus that increases or decreases cell activity.

2. The cell of claim 1, wherein the stimulus is light on the visible light spectrum.

3. The cell of claim 2, wherein the light is ultraviolet or infrared light.

4. The cell of claim 1, wherein said stimulus is blue, red, or infrared light.

5. The cell of claim 4, wherein the response to light is reversable.

6. A method for repairing a damaged nervous system in a patient in need thereof, the method comprising administration of the cells of any one of the preceding claim 1 and activating the response of said cells.

7. The method of claim 6, wherein the damage to the nervous system is caused by injury or disease.

8. A method for treating nervous system injury or disease in a patient in need thereof, the method comprising administering the cells of claim 1 and activating the response of said cells.

9. The method of claim 6, wherein the patient has damage to at least one of the brain or the spinal cord.

10. The method of claim 6, wherein the cells are obtained from at least one of the patient or a third-party donor and altered to have said response.

11. The method of claim 10, wherein the cells are obtained as optogenetic stem cells and differentiated to neural progenitor cells.

12. The method of claim 6, wherein administration of the cells improves motor recovery of the patient when compared to an untreated control.

13. (canceled)

14. (canceled)

15. (canceled)

16. (canceled)

17. (canceled)

18. The method of claim 6, wherein said response is activated by exposure to light.

19. The method of claim 6, wherein the cells are activated weeks or months after administration.

20. An in vitro method of generating a neural progenitor cell comprising
a) obtaining hPSCs from a donor;
b) plating said hPSCs from a) on mTeSR media and contacting said cells with CHIR and at least one Dual SMAD inhibitor (SMADi) and incubating said cells for about 5 days;
c) replating the cells from b) on Neural Induction Media and contacting said cells with CHIR, at least one Dual SMADi, and RA and incubating said cells for about 2 days;
d) contacting the cells from c) with RA, DAPT, and Pur(V2a) and incubating said cells for about 10 days; and
e) replating the cells from d) on BrainPhys Media and contacting said cells with G/BDNF, IGF, and CNTF to produce optogenetic neural progenitor cells.

21. The method of claim 20, wherein the neural progenitor cell is a V2a Spinal Interneuron.

22. The method of claim 20, wherein the cells of step b and step c are contacted with 0-6 μM, inclusive, of CHIR.

23. The method of claim 20, wherein the cells of step b and step c are contacted with 0, 2, 4, or 6 μM, of CHIR.

24. The method of claim 20, wherein the cells of step c and step d are contacted with 100 nM of RA.

25. The method of claim 20, wherein the cells of step d are contacted with 1 μM of DAPT.

26. The method of claim 20, wherein the cells of step d are contacted with 1 mM of Pur(V2a).
